(19) United States
(12) Patent Application Publication
(10) Pub. No.: US 2026/0036598 A1
(43) Pub. Date: Feb. 5, 2026

(54) BLOOD CELL AND FLUOROIMMUNOASSAY ANALYZER AND ANALYSIS METHOD THEREOF

(71) Applicant: B&E BIO-TECHNOLOGY CO., LTD., Yantai (CN)

(72) Inventors: Wenzheng LIU, Yantai (CN); Shijun SONG, Yantai (CN); Peng XU, Yantai (CN)

(73) Assignee: B&E BIO-TECHNOLOGY CO., LTD., Yantai (CN)

(21) Appl. No.: 18/968,958

(22) Filed: Dec. 4, 2024

(30) Foreign Application Priority Data
Jul. 30, 2024 (CN) 202411033388.5
Jul. 30, 2024 (CN) 202421824940.8

(51) Int. Cl. G01N 33/80; G01N 33/543

(52) U.S. Cl. CPC G01N 33/80; G01N 33/54386

(57) ABSTRACT

Disclosed are a blood cell and fluoroimmunoassay analyzer and an analysis method thereof. The blood cell and fluoroimmunoassay analyzer includes a rack, a whole blood sample assembly, an immunoreagent assembly, a blood cell counting assembly, an immunoassay assembly and a sampling assembly. The rack is provided with a first station, a second station, a third station and a fourth station at intervals along a first direction or a second direction. The whole blood sample assembly is provided at the first station. The immunoreagent assembly is provided at the second station. The blood cell counting assembly is provided at the third station and configured for classifying and counting the whole blood sample, and obtaining a hematocrit (HCT) value of the whole blood sample. The immunoassay assembly is provided at the fourth station and configured for performing a fluorescence immunoassay on the whole blood sample.

### CROSS-REFERENCE TO RELATED APPLICATIONS

The present application claims priority to Chinese Patent Application No. 202411033388.5, filed on Jul. 30, 2024, and Chinese Patent Application No. 202421824940.8, filed on Jul. 30, 2024, the entire contents of which are incorporated herein by reference.

### TECHNICAL FIELD

The present application relates to the technical field of medical device, and in particular to a blood cell and fluoroimmunoassay analyzer and an analysis method thereof.

### BACKGROUND

The blood cell analyzer and the dry fluoroimmunoassay analyzer are both conventional test devices in the laboratory. When it needs to perform blood cell analysis and immunoassay (such as C-reactive protein (CRP) or serum amyloid A (SAA), etc.) on one sample at the same time, two instruments are required to complete the task, which costs a lot of manpower and time. In addition, conventional fluoroimmunoassay analyzers cannot be adaptively adjusted according to different blood samples, resulting in certain deviations in the analysis results.

### SUMMARY

The main purpose of the present application is to provide a blood cell and fluoroimmunoassay analyzer and an analysis method thereof, aiming to solve the problem that the existing blood cell analysis and immunoassay operations are cumbersome and the deviations of the analysis results are relatively high.

To achieve the above purpose, the present application provides a blood cell and fluoroimmunoassay analyzer, including:
a rack provided with a first station, a second station, a third station and a fourth station at intervals along a first direction or a second direction;
a whole blood sample assembly provided at the first station and configured for storing a whole blood sample;
an immunoreagent assembly provided at the second station and configured for storing an immunoreagent;
a blood cell counting assembly provided at the third station and configured for classifying and counting the whole blood sample, and obtaining a hematocrit (HCT) value of the whole blood sample;
an immunoassay assembly provided at the fourth station and configured for performing a fluorescence immunoassay on the whole blood sample; and
a sampling assembly movably provided on the rack, where a movement stroke of the sampling assembly is configured to pass through the first station, the second station, the third station and the fourth station to transfer a corresponding sample or a reagent.

In an embodiment, the immunoassay assembly includes:
a reagent card installation portion provided at the fourth station and movably installed along the first direction, and configured to install a reagent card; and
a fluorescence read head portion provided at the fourth station and opposite to the reagent card on the reagent card installation portion, and configured to read a fluorescence intensity of the reagent card during a movement of the reagent card installation portion.

In an embodiment, the fluorescence read head portion is configured to move along the second direction, and is configured to move successively to a top of the reagent card in the second direction during the movement of the reagent card installation portion, so as to read fluorescence intensities of a plurality of reagent cards provided at intervals along the second direction.

In an embodiment, a plurality of the fluorescence read head portions are provided at intervals along the second direction, and each of the fluorescence read head portions is configured to one-to-one correspond with the reagent card on the reagent card installation portion.

In an embodiment, the immunoassay assembly further includes:
an installation seat provided at the fourth station;
a motor provided at the installation seat, where a motor rod of the motor is configured to extend along the first direction;
a movable block configured to connect the reagent card installation portion and the motor rod, so as to drive the reagent card installation portion to move along the first direction under a drive of the motor; and
at least one guiding rod configured to extend along the first direction and fixed to the installation seat, where an end of the guiding rod is configured to pass through the movable block.

In an embodiment, the immunoassay assembly further includes a detector provided at one side of the reagent card installation portion, and the detector is configured for detecting an installation status of the reagent card.

In an embodiment, the immunoassay assembly further includes a barcode scanner, and a scanning end of the barcode scanner is towards the reagent card installation portion, so as to scan a quick response (QR) code of the reagent card on the reagent card installation portion.

In an embodiment, the immunoassay assembly further includes:
a temperature measurement apparatus provided on the reagent card installation portion, and configured to detect a temperature of the reagent card; and
a heating apparatus provided on the reagent card installation portion and electrically connected to the temperature measurement apparatus, and configured to heat the reagent card according to a detection result of the temperature measurement apparatus.

In an embodiment, the immunoreagent assembly includes a reagent bottle provided at the second station, and a position of the reagent bottle is adjustable in the first direction or the second direction.

In an embodiment, the blood cell and fluoroimmunoassay analyzer further includes an optical mixing cup provided on the rack, and the optical mixing cup is configured for classifying a white blood cell sample in the blood cell counting assembly.

The present application further provides an analysis method of a blood cell and fluoroimmunoassay analyzer, based on the blood cell and fluoroimmunoassay analyzer as described above, including:
obtaining a hematocrit (HCT) value and a blood cell analysis result calculated by the blood cell counting assembly; and
controlling the immunoassay assembly to perform a fluorescence immunoassay on the whole blood sample in combination with the HCT value to obtain an immunoassay result.

In the technical solution of the present application, the whole blood sample assembly, the immunoassay assembly, the blood cell counting assembly and the immunoassay assembly are respectively provided at the first station, the second station, the third station and the fourth station at intervals, so that the blood cell analysis and the fluorescence immunoassay are integrated into one instrument, and the sample in blood cell analysis can be simultaneously applied to the immunoassay, saving costs and improving analytic efficiency. The transfer of samples or reagents is achieved through the sampling assembly, which is convenient for operation. Furthermore, the whole blood is the analytic target, and the hematocrit (HCT) value of the whole blood sample can be obtained through the blood cell counting assembly while performing the blood cell analysis, thereby correcting the influence of different blood samples on the immunoassay results, thereby improving the accuracy of the analysis results.

### BRIEF DESCRIPTION OF THE DRAWINGS

In order to illustrate the technical solutions in the embodiments of the present application or in the related art more clearly, the following briefly introduces the accompanying drawings required for the description of the embodiments or the related art. Obviously, the drawings in the following description are only part of embodiments of the present application. For those skilled in the art, other drawings can also be obtained according to the structures shown in these drawings without any creative effort.

FIG. 1 is a schematic structural view of a blood cell and fluoroimmunoassay analyzer according to an embodiment of the present application.

FIG. 2 is a schematic structural view of an immunoassay assembly in FIG. 1 according to an embodiment of the present application.

FIG. 3 is a schematic structural view of the immunoassay assembly in FIG. 1 according to an embodiment of the present application.

FIG. 4 is a schematic structural view of an immunoreagent assembly in FIG. 1.

FIG. 5 is a flow chart of an analysis method of the blood cell and fluoroimmunoassay analyzer according to an embodiment of the present application.

The realization of the objective, functional characteristics, and advantages of the present application are further described with reference to the accompanying drawings.

### DETAILED DESCRIPTION OF THE EMBODIMENTS

The technical solutions of the embodiments of the present application will be described in more detail below with reference to the accompanying drawings. It is obvious that the embodiments to be described are only some rather than all of the embodiments of the present application. All other embodiments obtained by those skilled in the art based on the embodiments of the present application without creative efforts shall fall within the scope of the present application.

It should be noted that if there are directional indications, such as up, down, left, right, front, back, etc., involved in the embodiments of the present application, the directional indications are only used to explain a certain posture as shown in the accompanying drawings. If the specific posture changes, the directional indication also changes accordingly.

In addition, if there are descriptions related to “first”, “second”, etc. in the embodiments of the present application, the descriptions of “first”, “second”, etc. are only for the purpose of description, and should not be construed as indicating or implying relative importance or implicitly indicates the number of technical features indicated. Thus, a feature delimited with “first”, “second” may expressly or implicitly include at least one of that feature. Besides, the meaning of “and/or” appearing in the application includes three parallel scenarios. For example, “A and/or B” includes only A, or only B, or both A and B. In addition, the technical solutions between the various embodiments can be combined with each other, but must be based on the realization by those skilled in the art. When the combination of technical solutions is contradictory or cannot be realized, it should be considered that the combination of such technical solutions does not exist or fall within the scope of protection claimed in the present application.

Blood cell analyzer and dry fluoroimmunoassay analyzer are both routine testing devices in the laboratory. When it needs to perform the blood cell analysis and the immunoassay (such as C-reactive protein (CRP) or serum amyloid A (SAA), etc.) on one sample at the same time, two instruments are usually required to complete the task, which costs a lot of manpower and time. In addition, conventional fluoroimmunoassay analyzer s cannot be adaptively adjusted according to different blood samples, resulting in certain deviations in the analysis results.

The main purpose of the present application is to provide a blood cell and fluoroimmunoassay analyzer and an analysis method thereof, aiming to solve the problem that the existing blood cell analysis and immunoassay operations are cumbersome and the deviations of the analysis results are relatively high.

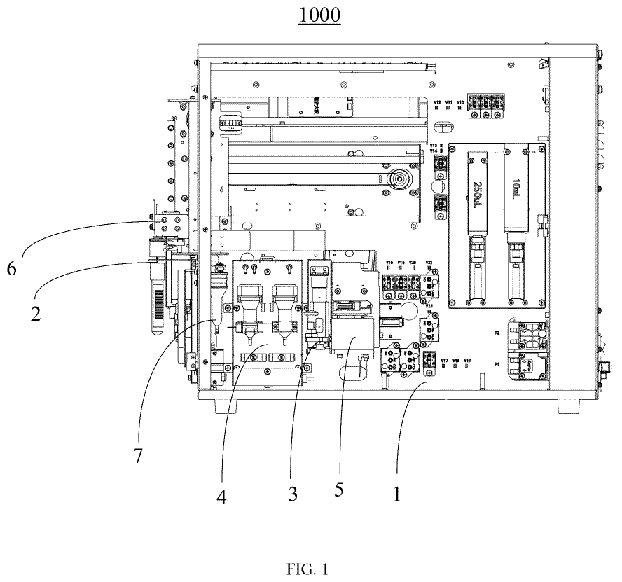

Referring to FIG. 1, the present application provides a blood cell and fluoroimmunoassay analyzer 1000, including a rack 1, a whole blood sample assembly 2, an immunoreagent assembly 3, a blood cell counting assembly 4, an immunoassay assembly 5, and a sampling assembly 6. The rack 1 is provided with a first station, a second station, a third station, and a fourth station provided at intervals along a first direction or a second direction. The whole blood sample assembly 2 is provided at the first station for storing the whole blood sample. The immunoreagent assembly 3 is provided at the second station for storing the immunoassay. The blood cell counting assembly 4 is provided at the third station for classifying and counting the whole blood sample and obtaining the hematocrit (HCT) value of the whole blood sample. The immunoassay assembly 5 is provided at the fourth station and configured for performing fluorescence immunoassay on the whole blood sample. The sampling assembly 6 is movably provided on the rack 1. The movable stroke of the sampling assembly 6 passes through the first station, the second station, the third station, and the fourth station for transferring the corresponding sample or reagent.

In the technical solution of the present application, the whole blood sample assembly 2, the immunoreagent assembly 3, the blood cell counting assembly 4 and the immunoassay assembly 5 are respectively provided at the first station, the second station, the third station and the fourth station provided at intervals, so that the blood cell analysis and the fluorescence immunoassay are integrated into one instrument, and the sample in the blood cell analysis can be simultaneously applied to the immunoassay, saving costs and improving the analytic efficiency. The samples or reagents can be transferred through the sampling assembly 6, which is convenient to operate. Furthermore, the whole blood is the analytic target, and the HCT value of the whole blood sample can be obtained through the blood cell counting assembly 4 while performing the blood cell analysis, so as to correct the influence of different blood samples on the immunoassay results, thereby improving the accuracy of the analysis results.

It should be noted that the HCT value of the whole blood sample refers to the volume percentage occupied by red blood cells in the blood, which can reflect the viscosity of the blood and the concentration degree of red blood cells. In some cases, changes in the HCT values may affect the results of the fluorescence immunoassays, especially when the analyte is present in plasma or serum, and changes in the concentration of red blood cells may affect the total volume of the sample and the dilution of the analyte. For example, if a patient is dehydrated, the HCT value will increase because the red blood cells are relatively concentrated due to the reduction in water. This may cause the concentration of the analyte in plasma or serum to appear higher than the actual level because the sample is concentrated by the red blood cells. Conversely, overhydration or hemodilution may also cause the HCT value to decrease, making the analyte concentration appear to be reduced. Therefore, when performing fluorescence immunoassays, if a whole blood sample is configured, the HCT value needs to be introduced to adjust the analysis results to ensure that the actual concentration of the analyte is accurately reflected.

It is worth mentioning that in a complete analysis process, the specific movement of the sampling assembly 6 includes: moving from the first station to the third station to transfer the whole blood sample to the blood cell counting assembly 4 for blood cell analysis and calculation of the HCT value, moving from the second station to the third station to mix the immunoassay with the whole blood sample in the red blood cell counting chamber to obtain the mixed reagent, and moving from the third station to the fourth station to transfer the mixed reagent to the immunoassay assembly 5 for immunoassay.

The blood cell counting assembly 4 includes: a white blood cell counting chamber, a red blood cell counting chamber, a DIFF chamber and an analyzer. The white blood cell counting chamber is configured to receive the whole blood sample, distribute white blood cells and red blood cells, and count the white blood cells. The red blood cell counting chamber is configured to receive the red blood cells distributed in the white blood cell counting chamber and count the red blood cells. The DIFF chamber is configured to classify and count the white blood cells. The analyzer integrates and analyzes the above data to obtain the blood cell analysis results and the HCT value of the whole blood sample.

It can be understood that, in the process of switching the sampling assembly 6 from transferring one reagent to transferring another reagent, in order to avoid contaminating the reagent in the container, the sampling assembly 6 needs to be cleaned.

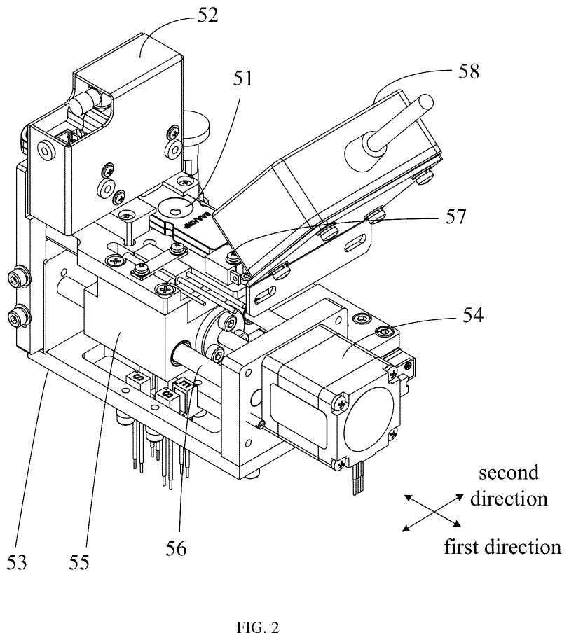

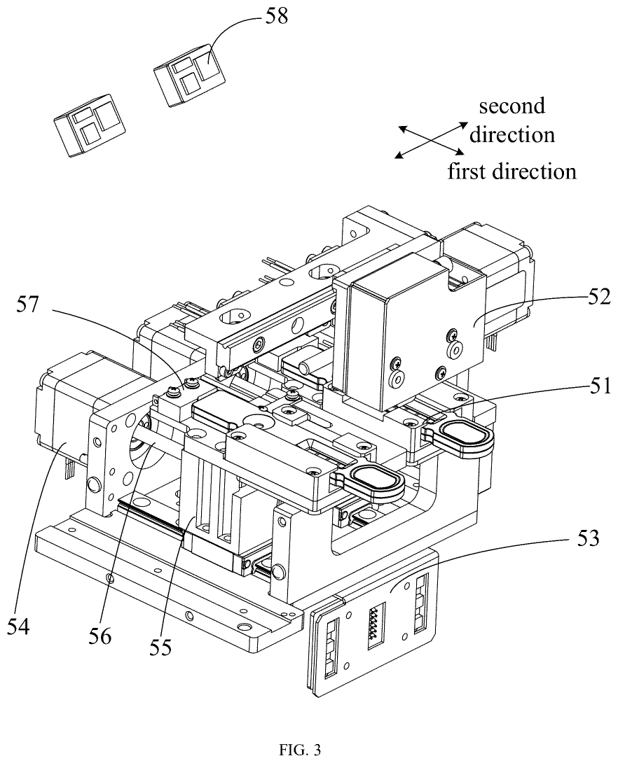

In order to realize the function of fluorescence immunoassay, specifically, referring to FIG. 2 and FIG. 3, the immunoassay assembly 5 includes a reagent card installation portion 51 and a fluorescence read head portion 52. The reagent card installation portion 51 is provided at the fourth station and can be movably installed along the first direction, and the reagent card installation portion 51 is configured to install the reagent card. The fluorescence read head portion 52 is provided at the fourth station and is provided relative to the reagent card on the reagent card installation portion 51, and is configured to read the fluorescence intensity of the reagent card when the reagent card installation portion 51 moves. In this way, by adopting the dry immunoassay strip to detect the immune parameter, it is convenient to use, and at the same time, the specific immunoassay does not need to be stored at low temperature, which also reduces the cost of reagent use and avoids reagent waste.

It is worth mentioning that in this embodiment, the fluorescence read head portion 52 is configured as a fluorescence generator and a fluorescence receiver. The excitation light source wavelength of the fluorescence generator is between 350 nm and 415 nm, and the wavelength of the fluorescence receiver receiving light is between 550 nm and 660 nm. When the excitation light is irradiated onto the reagent card, the fluorescence substance on the reagent card will be stimulated to emit light, forming T lines and C lines, which are converted into light of specific wavelengths through some optical paths to be detected. The immunoassay data can be obtained by comparing the relationship between T and C.

Referring to FIG. 3, in an embodiment of the present application, the fluorescence read head portion 52 can move along the second direction. The fluorescence read head portion 52 can move to the top of the reagent card in the second direction in sequence when the reagent card installation portion 51 moves, so as to read the fluorescence intensity of multiple reagent cards provided at intervals along the second direction. In this way, the reagent card installation portion 51 is provided with multiple clamping positions for reagent card installation, and the fluorescence read head portion 52 is synchronously provided, so that when the reagent card on a clamping position is replaced, the fluorescence read head portion 52 can read the reagent card on other clamping positions. Compared with the single-channel setting, the multi-channel scheme of this embodiment has higher efficiency of card reading.

It can be understood that the present solution does not limit the specific implementation form of the movement of the fluorescence read head portion 52. In an embodiment, the fluorescence read head portion 52 is connected to a motor rod of a motor, and the motor rod extends along the second direction. In an embodiment, the fluorescence read head portion 52 is connected to the output shaft of the stepping motor, thereby realizing the movement in the second direction.

In an embodiment, in order to further improve the card reading speed, a plurality of the fluorescence read head portions 52 are provided at intervals along the second direction, and each of the fluorescence read head portions 52 is configured to correspond to the reagent card on the reagent card installation portion 51. In this way, the synchronous progress of multiple reagent card reading processes can be realized, and the working efficiency is high.

In order to realize the movement of the reagent card installation portion 51, in an embodiment of the present application, referring to FIG. 2, the immunoassay assembly 5 also includes an installation seat 53, a motor 54, a movable block 55 and at least one guiding rod 56. The installation seat 53 is provided at the fourth station, the motor 54 is provided on the installation seat 53, and the motor rod of the motor 54 extends along the first direction. The movable block 55 connects the reagent card installation portion 51 and the motor rod, so as to drive the reagent card installation portion 51 to move along the first direction under the drive of the motor 54. The at least one guiding rod 56 extends along the first direction, the guiding rod 56 is fixed to the installation seat 53, and the end of the guiding rod 56 passes through the movable block 55. By providing the guiding rod 56, the movement direction of the movable block 55 is guaranteed, and the stability of the immunoassay assembly 5 is improved. It can be understood that the present solution does not limit the specific implementation form of the movement of the reagent card installation portion 51. In an embodiment, the movable block 55 is connected to the output shaft of the stepping motor to achieve the movement in the first direction.

In order to ensure that the reagent card can be inserted into the corresponding position, in an embodiment of the present application, the immunoassay assembly 5 also includes a detector 57 provided at one side of the reagent card installation portion 51 to detect the installation state of the reagent card. In this way, when the detector 57 detects that a reagent card is inserted, this information can be fed back to the host computer, indicating that this clamping position can be dripped with the mixed reagent, thereby improving the automation of the apparatus.

It can be understood that the present solution does not limit the specific implementation form of the detector 57. For example, in an embodiment, the detector 57 is configured as an infrared sensor. In an embodiment, the detector 57 is configured as a pressure sensor, and the user can adopt different strategies according to the actual situation.

In order to manage different samples in an information manner, in an embodiment of the present application, the immunoassay assembly 5 further includes a barcode scanner 58. The barcode scanning end on the barcode scanner 58 is towards the reagent card installation portion 51, and is configured to scan the QR code of the reagent card on the reagent card installation portion 51. By setting the barcode scanner 58, the information corresponding to the sample on the reagent card can be uploaded to the host computer, which is convenient for the background to summarize and store the data. It can be understood that the QR code on the reagent card stores information such as the tester, the test item, and the test time, which is not specifically limited here.

In an embodiment of the present application, the immunoassay assembly 5 further includes a temperature measurement apparatus and a heating apparatus. The temperature measurement apparatus is provided on the reagent card installation portion 51, and is configured to detect the temperature of the reagent card. The heating apparatus is provided on the reagent card installation portion 51 and is electrically connected to the temperature measurement apparatus, and is configured to heat the reagent card according to the test result of the temperature measurement apparatus. Since the treated test sample needs to be left to stand for 2-5 minutes for incubation after being quantitatively added to the sample adding position of the reagent card during immunoassay, the incubation temperature of the sample is controlled within a reasonable temperature range through the temperature measurement apparatus and the heating apparatus, thereby improving the incubation success rate of the sample and ensuring the reliability of the immunoassay result.

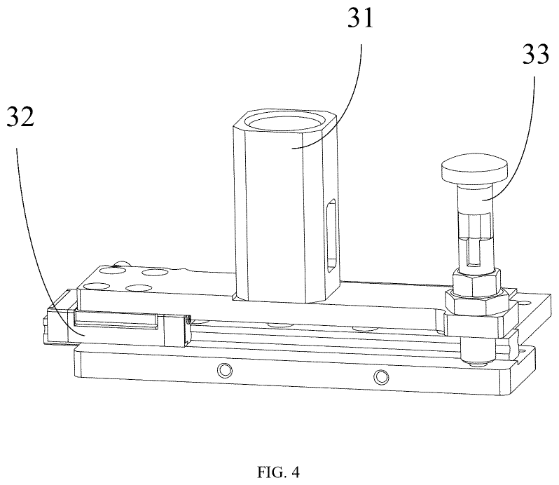

In an embodiment of the present application, referring to FIG. 4, the immunoreagent assembly 3 includes a reagent bottle 31 provided on the second station, and the position of the reagent bottle 31 in the first direction or the second direction is adjustable, which is convenient to accommodate without using the immunoassay, saving space. Specifically, the immunoreagent assembly 3 is also provided with a sliding cooperation structure 32 and a limiting member 33, and the reagent bottle 31 is provided on the sliding cooperation structure 32 to slide under the guidance of the sliding cooperation structure 32. The limiting member 33 is provided on the third station to limit the sliding stroke of the reagent bottle 31. The driving mode of the reagent bottle 31 may be manual or electrically controlled by the driving apparatus, which is not limited here. The limiting member 33 may be a positioning pin provided on the sliding cooperation structure 32 or a stopper provided on the third station, which is not specifically limited here.

In an embodiment of the present application, referring to FIG. 1, the blood cell and fluoroimmunoassay analyzer 1000 further includes an optical mixing cup 7 provided on the rack 1, and the optical mixing cup 7 is configured to classify the white blood cell sample in the blood cell counting assembly 4. The whole blood and DIFF reagent need to be added into the optical mixing cup 7, and get mixed. The mixed sample will enter the optical component, and the optical classification of white blood cells will be carried out by laser sheath flow technology, thereby achieving the classification of the white blood cell sample, thus providing raw data for blood cell analysis. It can be understood that the sampling assembly 6 can be moved to the optical mixing cup 7 to transfer the white blood cell sample therein for classification.

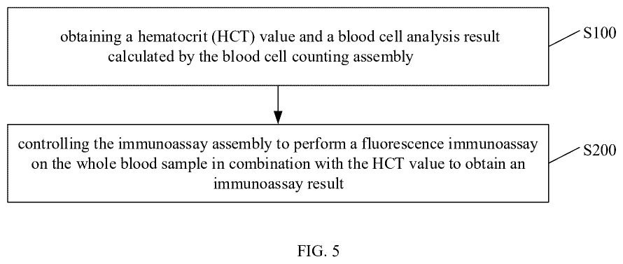

The present application also provides an analysis method of a blood cell and fluoroimmunoassay analyzer. Referring to FIG. 5, the analysis method of the blood cell and fluoroimmunoassay analyzer includes the following steps.

S100: obtaining the HCT value and blood cell analysis result calculated by the blood cell counting assembly 4.

S200: controlling the immunoassay assembly 5 to perform fluorescence immunoassay on the whole blood sample in combination with the HCT value to obtain the immunoassay result.

The technical solution of the present application calculates the HCT value while performing blood cell analysis, and the HCT value can be obtained by the red blood cell counting, which is more convenient. By performing fluorescence immunoassay on the whole blood sample in combination with the HCT value, the actual concentration of the analyte is ensured, thereby correcting the influence of different blood samples on the immunoassay result, and improving the accuracy of the analysis result.

The above descriptions are only embodiments of the present application, and are not intended to limit the scope of the present application. Under the inventive concept of the present application, any equivalent structural transformations made by using the contents of the description and drawings of the present application, or direct/indirect applications in other related technical fields are included in the scope of the present application.

What is claimed is:

1. A blood cell and fluoroimmunoassay analyzer, comprising:
a rack provided with a first station, a second station, a third station and a fourth station at intervals along a first direction or a second direction;
a whole blood sample assembly provided at the first station and configured for storing a whole blood sample;
an immunoreagent assembly provided at the second station and configured for storing an immunoreagent;
a blood cell counting assembly provided at the third station and configured for classifying and counting the whole blood sample, and obtaining a hematocrit (HCT) value of the whole blood sample;
an immunoassay assembly provided at the fourth station and configured for performing a fluorescence immunoassay on the whole blood sample; and
a sampling assembly movably provided on the rack, wherein a movement stroke of the sampling assembly is configured to pass through the first station, the second station, the third station and the fourth station to transfer a corresponding sample or a reagent.

2. The blood cell and fluoroimmunoassay analyzer according to claim 1, wherein the immunoassay assembly comprises:
a reagent card installation portion provided at the fourth station and movably installed along the first direction, and configured to install a reagent card; and
a fluorescence read head portion provided at the fourth station and opposite to the reagent card on the reagent card installation portion, and configured to read a fluorescence intensity of the reagent card during a movement of the reagent card installation portion.

3. The blood cell and fluoroimmunoassay analyzer according to claim 2, wherein the fluorescence read head portion is configured to move along the second direction, and is configured to move successively to a top of the reagent card in the second direction during the movement of the reagent card installation portion, so as to read fluorescence intensities of a plurality of reagent cards provided at intervals along the second direction.

4. The blood cell and fluoroimmunoassay analyzer according to claim 2, wherein a plurality of the fluorescence read head portions are provided at intervals along the second direction, and each of the fluorescence read head portions is configured to one-to-one correspond with the reagent card on the reagent card installation portion.

5. The blood cell and fluoroimmunoassay analyzer according to claim 2, wherein the immunoassay assembly further comprises:
an installation seat provided at the fourth station;
a motor provided at the installation seat, wherein a motor rod of the motor is configured to extend along the first direction;
a movable block configured to connect the reagent card installation portion and the motor rod, so as to drive the reagent card installation portion to move along the first direction under a drive of the motor; and
at least one guiding rod configured to extend along the first direction and fixed to the installation seat, wherein an end of the guiding rod is configured to pass through the movable block.

6. The blood cell and fluoroimmunoassay analyzer according to claim 2, wherein the immunoassay assembly further comprises a detector provided at one side of the reagent card installation portion, and the detector is configured for detecting an installation status of the reagent card.

7. The blood cell and fluoroimmunoassay analyzer according to claim 2, wherein the immunoassay assembly further comprises a barcode scanner, and a scanning end of the barcode scanner is towards the reagent card installation portion, so as to scan a quick response (QR) code of the reagent card on the reagent card installation portion.

8. The blood cell and fluoroimmunoassay analyzer according to claim 2, wherein the immunoassay assembly further comprises:
a temperature measurement apparatus provided on the reagent card installation portion, and configured to detect a temperature of the reagent card; and
a heating apparatus provided on the reagent card installation portion and electrically connected to the temperature measurement apparatus, and configured to heat the reagent card according to a detection result of the temperature measurement apparatus.

9. The blood cell and fluoroimmunoassay analyzer according to claim 1, wherein the immunoreagent assembly comprises a reagent bottle provided at the second station, and a position of the reagent bottle is adjustable in the first direction or the second direction.

10. The blood cell and fluoroimmunoassay analyzer according to claim 1, further comprising:
an optical mixing cup provided on the rack,
wherein the optical mixing cup is configured for classifying a white blood cell sample in the blood cell counting assembly.

11. An analysis method of a blood cell and fluoroimmunoassay analyzer, based on the blood cell and fluoroimmunoassay analyzer according to claim 1, comprising:
obtaining the HCT value and a blood cell analysis result calculated by the blood cell counting assembly; and
controlling the immunoassay assembly to perform the fluorescence immunoassay on the whole blood sample in combination with the HCT value to obtain an immunoassay result.
